(19) United States
(12) Patent Application Publication
(10) Pub. No.: US 2024/0342394 A1
(43) Pub. Date: Oct. 17, 2024

(54) Syringe Having Combined Luer Lock And Pen Needle Attachment Capability

(71) Applicant: Becton, Dickinson and Company, Franklin Lakes, NJ (US)

(72) Inventor: Des Whitney, Aughrim, Wicklow (IE)

(73) Assignee: Becton, Dickinson and Company, Franklin Lakes, NJ (US)

(21) Appl. No.: 18/683,670

(22) PCT Filed: Sep. 3, 2021
(86) PCT No.: PCT/US2021/049099

§ 371 Date: Feb. 14, 2024

(51) Int. Cl. A61M 5/34; A61M 5/31; A61M 39/10

(52) U.S. Cl. CPC A61M 5/345; A61M 5/3134; A61M 2039/1077; A61M 2039/1088

(57) ABSTRACT

A male needless connector having a tapered elongate tip and a collar compliant with ISO standards, the collar having an outer sidewall surface with a plurality of threads for connection to corresponding inner threads of a pen needle. The male needleless connector having a taper between a distal end of the elongate tip and an inner sidewall of the elongate tip. The male needless connector being configured to receive a pen needle.

### TECHNICAL FIELD

The present disclosure is the field of medication delivery devices, and in particular, the disclosure is directed to male Luer connectors having cross-compatibility with female Luer connectors and small-gauge pen needles.

### BACKGROUND

Medication pens are typically used to inject a small-dose of medication into a patient. A person who must periodically self-inject doses of medication will typically carry a medication pen and several single-use pen needles. Medical practitioners can also utilize medication pens for withdrawing medication from a vial and injecting the medication with a medication pen and an accompanying pen needle.

Pen needles are commonly adapted to achieve injection to a specified target depth in a subcutaneous region (SC) region and commonly have shorter needle cannula lengths (such as 4 mm and 5 mm needles) and smaller needle cannula gauges (such as 32 g). In applications where a practitioner has to withdraw medication from a vial having a rubber stopper, pen needles are often not capable of sufficiently piercing through the stopper due to their shorter needle cannula length. In further instances, pen needles can bend, buckle or otherwise become damaged when piercing the stopper to their smaller needle cannula gauge. In applications where a greater volume of medication is required to be aspirated from the vial, pen needles can often require significantly more time to withdraw, as well as greater withdrawal forces due to their smaller needle cannula gauge.

Thus, there is a need in the art to provide a medication delivery device compatible with pen needles which can quickly aspirate fluids from a vial for use in SC region injections.

### SUMMARY

A first aspect of the present disclosure relates to a male needless connector comprising an elongate tip having a distal end and a proximal base, a tapered outer surface and a lumen extending through the elongate tip, the proximal base having a diameter greater than a diameter of the distal end the lumen defining an inner sidewall; and, a collar having a substantially cylindrical shape and a distal end, the collar partially surrounding the elongate tip, the collar having a cavity forming an inner sidewall surface and an outer sidewall surface, the inner sidewall surface having a diameter greater than the proximal base of the elongate tip, the outer sidewall surface including a plurality of threads for connection to corresponding inner threads of a pen needle. The inner sidewall surface of the collar includes inner threads for connection to corresponding threads of a female luer connector.

In some embodiments, a female luer connector is inserted in between the elongate tip and inner sidewall surface.

In some embodiments, the male needless connector further comprises a taper between the inner sidewall of the elongate tip and the distal end of the elongate tip. In some embodiments, the taper has a flat surface. In some embodiments, the taper has a flat surface with rounded edges. In some embodiments, the taper has an angle θ relative to a central axis Z of the male needleless connector. In some embodiments, the angle θ is in the range of 26 degrees to 28.5 degrees. In some embodiments, the taper is configured to create a seal with a post of a pen needle.

In some embodiments, the male needless connector is compatible with ISO-80369 standards for Male Luer Locks with Fixed Collars. In some embodiments, a projection distance G′ of the distal end of the elongate tip from the proximal base is 7.5 mm and a projection distance M′ of the distal end of the elongate tip from the distal end of the collar is 2.1 mm. In some embodiments, a projection distance G′ of the distal end of the elongate tip from the proximal base is 7.5 mm and a projection distance M′ of the distal end of the elongate tip from the distal end of the collar is in the range of 4.00 mm to 4.80 mm. In some embodiments, a projection distance G′ of the distal end of the elongate tip from the proximal base is 7.5 mm and a projection distance M′ of the distal end of the elongate tip from the distal end of the collar is in the range of 2.1 mm to 4.80 mm. In some embodiments, the plurality of threads are configured to create more than 2 turns with inner threads of a standard pen needle.

Further embodiments of the present disclosure are directed to a kit comprising a syringe having a barrel with an open proximal end, a distal end and a cavity, the male needless connector, where the male needless connector integrally molded to the distal end of the barrel; a pen needle hub having a double-ended cannula for coupling to a standard pen needle assembly, the pen needle having a body and side wall forming an open end, the side wall having inner threads for coupling to a standard pen needle assembly; and, a standard female luer needle having a needle hub and a cannula, the needle hub having a proximal end and a distal end, the cannula extending through the distal end.

In some embodiments, the body has a distal face with a peripheral edge forming a shoulder, from the shoulder extends a tower away from the open end, the shoulder having a shoulder sidewall and a distal end wall. In some embodiments, a post projects proximally from an inner face of the end wall of the pen needle for supporting the double-ended cannula, the post having a conical shaped base portion. In some embodiments, a taper between the inner sidewall of the elongate tip and the distal end of the elongate tip is configured to create a seal with the conical shaped base portion of the post. Some embodiments, the conical shaped base portion of the post is at least partially inserted within the lumen of the elongate tip creating a seal.

In some embodiments, the plurality of threads of the outer sidewall surface of the collar are configured to interdigitate with the inner threads of the pen needle until an inner surface of the shoulder abuts the distal end of the collar.

In some embodiments, the cannula of the standard female luer needle is a standard-gauge needle and the double-ended needle cannula of the pen needle is a small-gauge needle.

Further embodiments are directed to a method of use of the kit comprising the steps of: attaching the needle hub to the male needless connector; inserting the cannula into a stopper of a vial; aspirating medicine from a vial by withdrawing a plunger rod of the barrel; removing the needle hub from the male needless connector; and, attaching the pen needle to the male needless connector. Wherein the pen needle is attached to the male needless connector by threading the inner threads of the pen needle to the plurality of threads of the outer sidewall surface of the collar of the male needless connector.

### BRIEF DESCRIPTION OF THE DRAWINGS

FIG. 1 illustrates a standard ISO compatible male luer connector;

FIG. 2 illustrates a standard ISO compatible pen needle;

FIG. 3 illustrates a syringe and a male needleless connector in accordance with one or more embodiments of the present disclosure;

FIG. 4 illustrates a detailed view of a male needleless connector in accordance with one or more embodiments of the present disclosure;

FIG. 5 illustrates a bottom perspective view of a pen needle in accordance with one or more embodiments of the present disclosure;

FIG. 6 illustrates a front perspective view of a pen needle in accordance with one or more embodiments of the present disclosure;

FIG. 7 illustrates a cross-sectional view of a pen needle in accordance with one or more embodiments of the present disclosure;

FIG. 8 illustrates a cross-sectional view of male luer connector connected to a pen needle in accordance with one or more embodiments of the present disclosure;

FIG. 9 illustrates a detailed view A of the cross-sectional view of FIG. 9 in accordance with one or more embodiments of the present disclosure;

FIG. 10 illustrates a cross-sectional view of an ISO compatible pen needle and an ISO compatible male needleless connector in accordance with one or more embodiments of the present disclosure; and,

FIG. 11 illustrates a kit having a pen needle, a standard female needle cannula and a male needleless connector in accordance with one or more embodiments of the present disclosure.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

For purposes of the description hereinafter, the terms “proximal”, “distal”, “longitudinal”, and derivatives thereof shall relate to the disclosure as it is oriented in the drawing figures. However, it is to be understood that the disclosure may assume alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

As used herein, the use of “a,” “an,” and “the” includes the singular and plural.

As used herein, the term “medical device” refers to common medical devices having threaded or interlocking connections, the connections having corresponding mating elements. By way of example but not limitation, a syringe may have a threaded connection which releasably interlocks with a secondary medical device such as a needless connector of a catheter, an IV line and the like. The threaded connection may include a lumen defining a fluid path surrounded by a protruding wall having the threaded means for attaching to the secondary medical device.

As would be readily appreciated by skilled artisans in the relevant art, while descriptive terms such as “thread”, “taper”, “tab”, “wall”, “proximal”, “side”, “distal” and others are used throughout this specification to facilitate understanding, it is not intended to limit any components that can be used in combinations or individually to implement various aspects of the embodiments of the present disclosure.

As used herein, the term “Luer connector” or “Luer Lock Connector” refers to a connection collar that is the standard way of attaching syringes, catheters, hubbed needles, IV tubes, etc. to each other. The Luer connector consists of one or more interlocking tubes, slightly tapered to hold together with just a simple pressure/twist fit/friction fit. Luer connectors can optionally include an additional outer rim of threading, allowing them to be more secure. The Luer connector can interlock and connect to the end located on the vascular access device (VAD). A Luer connector comprises a profiled center passageway for fluid communication from the chamber of the barrel of a syringe to the hub of a VAD extending in a distal direction, a cylindrical outer wall surrounding the profiled center passageway, a distal end and a proximal end. A Luer connector also has a distal end channel that releasably attaches the Luer connector to the hub of a VAD, and a proximal end channel that releasably attaches the Luer connector to the barrel of a syringe. As used herein, the term “Luer connector” refers to a male luer connector or a female luer connector.

As used herein, the term “male luer connector” or “male needless connector” refers to a luer connector having a profiled center passageway (a tapered elongate tip) and a cylindrical outer wall (a collar).

As used herein, the term “standard” when used with one or more of the connectors described herein is referred to a connector which is compliant with the particular International Organization for Standardization (ISO) standards for said connectors.

As used herein, the term “pen injector” refers to a standard pen injector which complies with ISO 11608-1:2014. ISO 11608-1:2014 specifies requirements and test methods for needle-based injection systems (NISs) intended to be used with needles and with replaceable or non-replaceable containers. As used herein, the term “pen needle” refers to a single-use, double-ended, sterile needle for needle-based injection systems (NISs) that fulfil the specifications of ISO 11608-1. Pen needles as described in the present disclosure comply with ISO 11608-2:2012 unless stated otherwise. ISO 11608-2:2012 specifies requirements and test methods for single-use, double-ended, sterile needles for needle-based injection systems (NISs) that fulfill the specifications of ISO 11608-1.

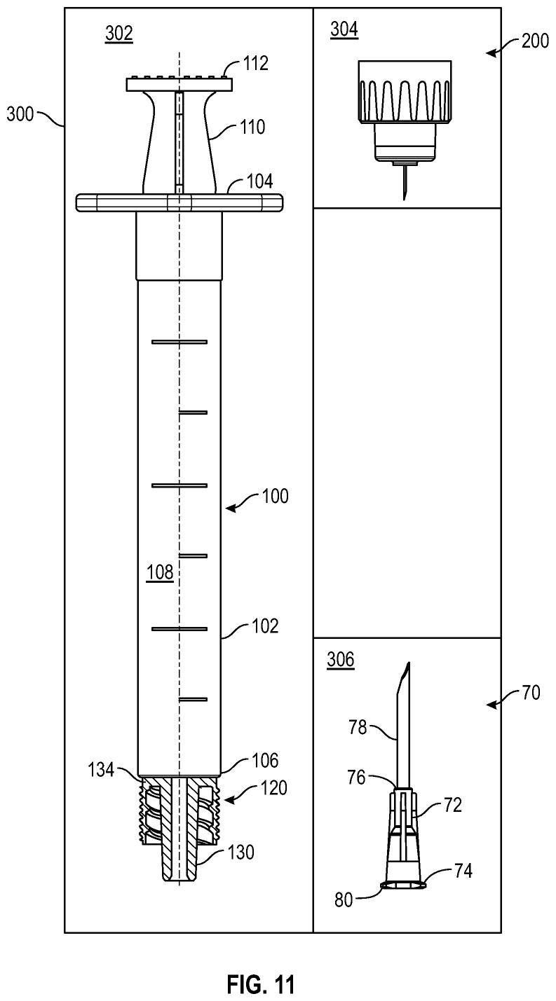

Connectors used with drug delivery devices typically share a common ISO standard luer connection. A standard male luer tip or standard male connector has specifications in accordance with ISO 594-1:1986 and 594-2:1998 and recently published ISO-80369-2021 standards. Said standard male luer connectors are configured to interdigitate and connect with standard female luer connectors that can commonly be found on luer needle hubs (such as standard female luer needle 70 shown in FIG. 11). Conventional pen needles are specified under ISO-11608-2:2012 to specifically to be compatible with pen injectors under ISO 11608-1, however, conventional pen needles are not compatible with standard male luer connectors.

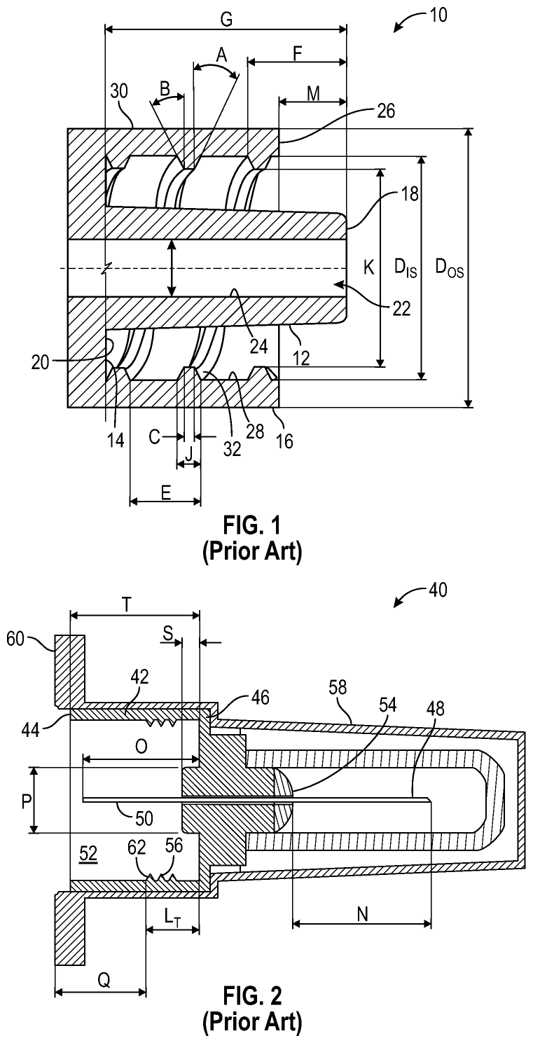

FIG. 1 illustrates a standard male luer lock connector 10 in accordance with recently published ISO-80369-2021 for Male Luer Locks with Fixed Collars. The standard male luer lock connector 10 comprises a tapered elongate tip 12 extending from a proximal wall 14 and a collar 16 extending from the proximal wall 14 and partially surrounding the elongate tip 12.

The elongate tip 12 has a trapezoidal cross-section with a distal end 18 and a proximal base 20. The proximal base 20 has a diameter which is greater than a diameter of the distal end 18 of the elongate tip 12. The elongate tip 12 has an outer surface which is tapered and decreases in diameter from the proximal base 14 to the distal end 18. The elongate tip 12 further comprises a lumen 22 extending through the elongate tip 12 and in fluid communication with a cavity of a syringe barrel. The lumen 22 defines an inner sidewall surface 24 of the elongate tip 12, the inner sidewall surface 24 extending from the distal end 18 to the proximal base 20.

The collar 16 has a substantially cylindrical shape and extends from the proximal wall 14 to a distal end 26 of the collar 16. The collar 16 has a length which is less than a length of the elongate tip 12. The collar 16 has an inner sidewall 28 and an outer sidewall 30, the inner sidewall 28 having a diameter which is greater than the diameter of the proximal base 20 of the elongate tip 12, and the outer sidewall 30 having a diameter greater than the diameter of the inner sidewall 28. The inner sidewall 28 and outer sidewall 30 define a thickness of the collar 16. The inner sidewall 28 includes inner threads 32 for connection to corresponding threads of a female luer connector. In the illustrated standard male luer lock connector 10, the inner sidewall surface 24 defined by the lumen 22 is a right angle to the distal end 18 of the elongate tip 12.

The dimensioned characteristics A (angle of internal thread profile on non-bearing surface), B (angle of internal thread profile on bearing surface), C (width of thread profile at crest), E (nominal pitch), F (distance from the distal end 18 of the elongate tip to the bottom of the first complete thread profile of the internal thread, which is an auxiliary dimension), J (width of the thread profile at root) and K (minor inside diameter) of the inner threads 32 are defined under Luer Slip and Luer Lock ISO-80369 standards and have specific value ranges (minimum, nominal and maximum dimensions) for said dimensions. Of relevance to the present disclosure, the ISO-80369 standards also have specific value ranges for the diameter Dis of the inner sidewall 28, the diameter Dos of the outer sidewall 30, diameter D_{L} of the lumen 22, as well as the projection distance G of the distal end 18 of the elongate tip 12 from the proximal wall 14, and the projection distance M of the distal end 18 of the elongate tip 12 from the distal end 26 of the collar. In some embodiments, the aforementioned dimensions are compliant with ISO-80369 standards. Under ISO-80369 standards, the projection distance G is in the range of 7.5 mm to 10.5 mm, the projection distance M has a minimum value of 2.1 mm, the diameter DI, has a maximum value of 2.9 mm, the diameter Dos is in the range of 8.8 mm to 11.5 mm, the elongate tip 130 has a 6% taper with a tip diameter of 3.97 mm to 4.72 mm at the distal end 132 of the elongate tip 130 and a base diameter of 4.375 mm to 4.477 mm at the proximal base 134 of the elongate tip 130.

FIG. 2 illustrates a standard pen needle 40 in accordance with ISO 11608-2:2012 standard for pen needles. The standard pen needle 40 comprises a needle hub 42 having an open proximal end 44, a distal wall 46 and a double-ended needle cannula 48 having a proximal end 50 within a cavity 52 of the needle hub 42 and a distal end 54 extending from the distal wall 46 of the needle hub 42 in a distal direction. The needle hub 42 further comprises threads 56 disposed on an inner wall of the needle hub 42. The standard pen needle 40 in accordance with ISO 11608-2:2012 can further include a needle container 58 for fully enclosing the needle, the needle container 58 having an open proximal end 60.

As shown in FIG. 2, the dimensioned characteristics N (length of needle cannula from the distal end 54), O (length of needle cannula to the distal wall 46 within the cavity 52), P ( ) Q (distance from proximal end 62 of the threads 56 to the open proximal end 60 of the needle container 58) and S (projection distance of post within the cavity) are defined under ISO 11608-2:2012 standards for pen needles and have specific value ranges (minimum, nominal and maximum dimensions) for said dimensions. Of relevance to the present disclosure, the ISO 11608-2:2012 standards also have specific value ranges for a length L_{T} of a proximal end 62 of the threads 56 from the distal wall 46, a length Q of the proximal end 62 of the threads 56 to the open proximal end 60 of the needle container 58, And a length T from an inner surface of the distal wall 46 to the open proximal end 44. In some embodiments, the aforementioned dimensions are compliant with ISO 11608-2:2012 standards. Under ISO 11608-2:2012 standards, the length L_{T} is less than 3.7 mm, the length Q is less 6.0 mm and the length T is less 7.5 mm.

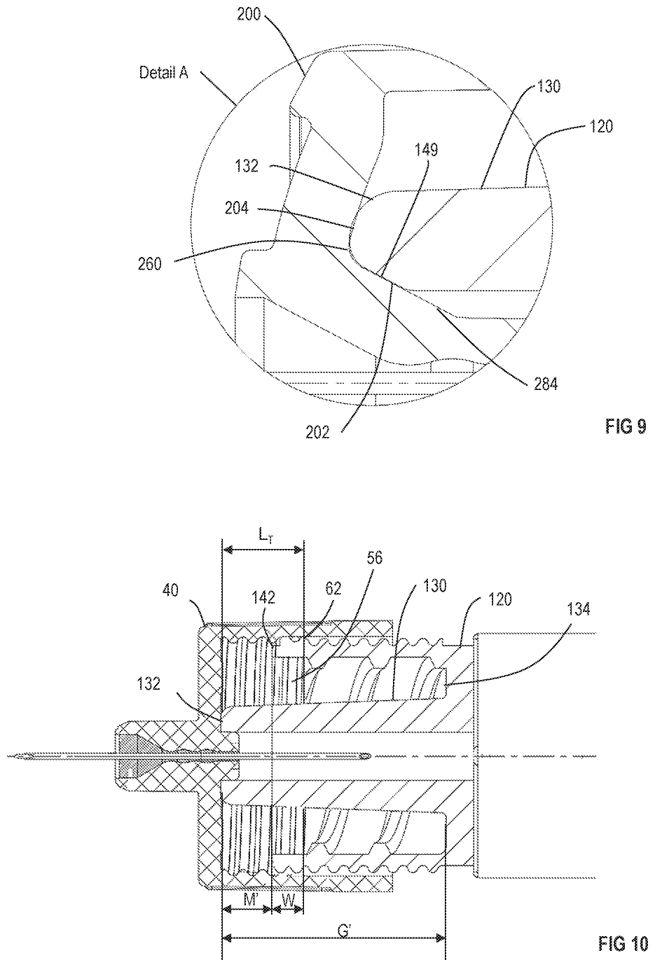

Compliance with ISO-80369 standards ensures that corresponding standard female connectors (such as a standard female luer needle 70 shown in FIG. 11) can connect with the male needless connector 10 by either creating an interference fit with the elongate tip 12, or by threading onto the inner threads 32, or both. However, as shown in FIG. 10 and as discussed in greater detail below, a standard needle pen 40 cannot sufficiently lock with the standard male luer lock connector 10.

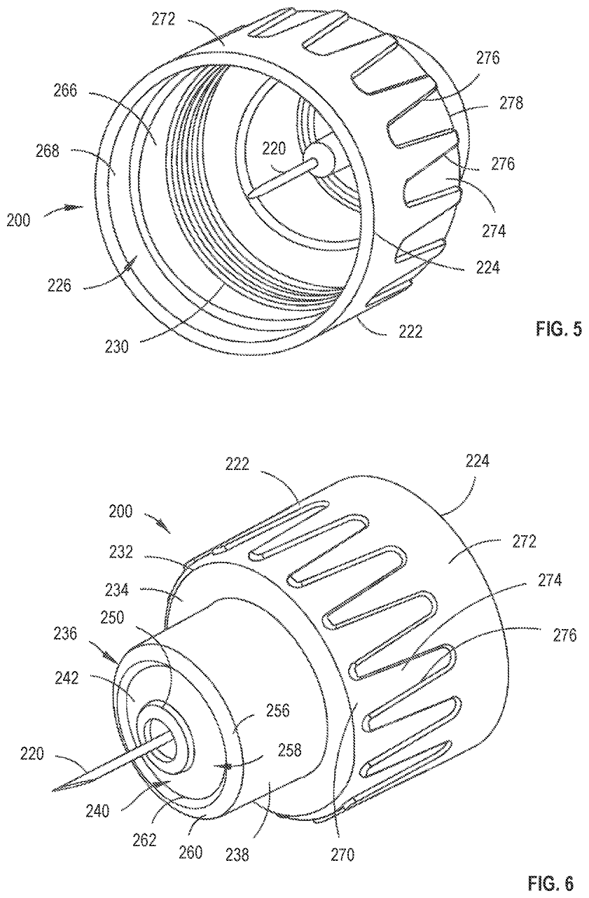

Embodiments of the present disclosure are directed to a modified male Luer Lock connector 120 which is compatible with a conventional pen needle (such as the pen needle 200 of FIGS. 5 though 7) while still maintaining compatibility with standard female Luer lock connectors under ISO standards (such as standard female luer needle 70 shown in FIG. 11). In some embodiments, the pen needle is compatible with ISO 11608 standards. In some embodiments, the pen needle is a BD NANO™ PRO 4 MM PEN NEEDLE by BECTON DICKINSON AND CO. In some embodiments, the male Luer Lock connector 120 is molded onto a conventional syringe.

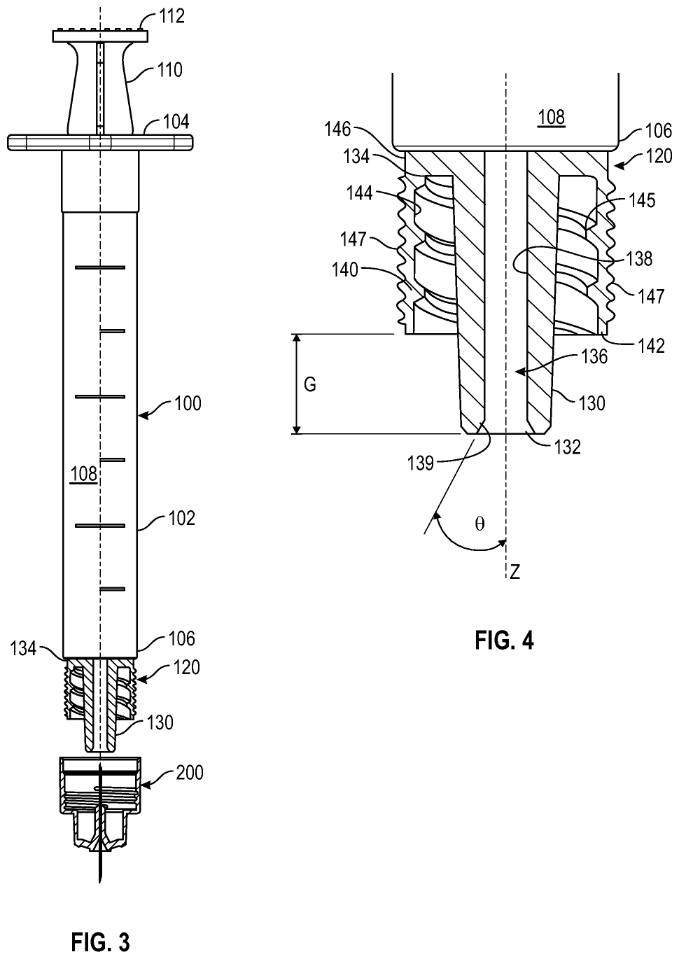

As previously set forth, FIGS. 1 and 2 illustrate prior art devices under ISO standards. FIGS. 3 and 4 illustrate embodiments of a syringe 100 having a male needleless connector 120 which is compatible and compliant with ISO 80369-7:2021 standard, and thus incorporates all of the dimensions of the standard male luer lock connector 10. The male needleless connector 120 is configured to receive a standard female Luer lock connector such as a needle hub and cannula (such as standard female luer needle 70 shown in FIG. 11), as well as a pen needle 200 as described herein.

The syringe 100 comprises a barrel 102 and a plunger rod 110. The barrel 102 has an open proximal end 104, a distal end 106 and a cavity 108. The plunger rod 110 has a distally positioned thumb press 112. As shown in FIG. 3 and as shown in greater detail in FIG. 4, the male needless connector 120 comprises a tapered elongate tip 130 extending from the distal end 106 of the barrel 102 and a collar 140 extending from the distal end 160 of the barrel 102 and partially surrounding the elongate tip 130.

The elongate tip 130 has a trapezoidal cross-section with a distal end 132 and a proximal base 134. As shown in the figures, the proximal base 134 is unitary with the distal end 106 of the barrel 102. The proximal base 134 has a diameter greater than a diameter of the distal end 132 of the elongate tip 130. The elongate tip 130 has an outer surface which is tapered and decreases in diameter from the proximal base 134 to the distal end 132. The elongate tip 130 further comprises a lumen 136 extending through the elongate tip 130 and in fluid communication with the cavity 108 of the barrel 102. The lumen 136 defines an inner sidewall 138 of the elongate tip 130, the inner sidewall extending from the distal end 132 to the proximal base 134.

The collar 140 has a substantially cylindrical shape and extends from the distal end 106 of the barrel 102 to a distal end 142 of the collar 140. The collar 140 has a length which is less than a length of the elongate tip 130. The collar 142 has a cavity forming an inner sidewall surface 144 and an outer sidewall surface 146, the inner sidewall surface 144 having a diameter which is greater than the diameter of the proximal base 134 of the elongate tip 130, and the outer sidewall surface 146 having a diameter greater than the diameter of the inner sidewall surface 144. The inner sidewall surface 144 and outer sidewall surface 146 define a thickness of the collar 140. The inner sidewall surface 144 includes inner threads 145 for connection to corresponding threads of a female luer connector. In some embodiments, the female luer connector is a standard female luer needle 70 shown in FIG. 11

Referring back to FIG. 1, the dimensions as previously described herein apply to the male needleless connector 120 as both the standard male luer lock connector 10 of FIG. 1 and the male needleless connector 120 are compatible and compliant with ISO standard 80369-7:202.

As shown in FIG. 4, the outer sidewall surface 146 includes a plurality of threads 147. The plurality of threads 147 in some embodiments have a pitch of 0.8 mm, a thread angle of 60 degrees and a distance from root to crest of 0.293 mm per ISO 11608 standards. Furthermore, in some embodiments the plurality of threads 147 have a root diameter of 0.2 mm and a crest diameter of 0.2 mm. Because ISO luer standards require the corresponding connector to be inserted in between the elongate tip 130 and the inner sidewall surface 144 of the collar, having a plurality of threads 147 on the outer sidewall surface 146 of the collar 140 does not hinder connection of a corresponding connector. Furthermore, in some embodiments, the distal end 132 of the elongate tip 130 has a taper 139 between the inner sidewall 138 of the elongate tip 130 and the distal end 132 of the elongate tip 130. In some embodiments, as best shown in FIG. 9, the taper 139 has a flat surface 149 which has rounded edges. In some embodiments, the taper 139 has an angle θ relative to a central axis Z of the syringe 100. In some embodiments, the flat surface 149 has an angle θ relative to the central axis Z of the syringe 100. In some embodiments, the angle θ is in the range of 26 degrees to 28.5 degrees. As explained in further detail below, the flat surface 149 and the taper 139 is configured to create a seal with a post 244 of a pen needle 200 as explained in further detail below. Female luer connectors create a interference fit and a fluid-tight seal with the elongate tip 130, however female luer connectors do not interact with the inner sidewall 138 of the elongate tip 130 and, and do not create a seal with the distal end 132 of the elongate tip 130, thus, having a taper 139 on the inner sidewall 138 of the elongated tip 130 does not hinder connection of a corresponding connector. Stated differently, the seal created between the taper 138 and the post 244 of the pen needle 200 (as explained in further detail below) does not affect compatibility of standard female luer connectors.

Referring to FIGS. 3 and 5-7, a pen needle hub 200 includes a double-ended cannula 220 beveled and sharpened at both ends for coupling to a standard pen needle assembly and for penetrating the skin of the patient. The pen needle hub 200 has a body 222 having a side wall 224 to form an open end 226. In the embodiment shown, body 222 has a substantially cylindrical shape. The open end 226 forms an internal cavity with internal threads 230 disposed on an inner surface of the side wall 224 for coupling to the pen needle delivery device.

Body 222 of pen needle hub 200 has a distal face 260 with a peripheral edge 232 forming a shoulder 234. The shoulder 234 can be oriented in a plane substantially perpendicular to a central axis of the pen needle hub 200. A tower 236 forming an upper end portion of pen needle hub 200 extends from shoulder 234 in the direction of the central axis away from the open end 226. The tower 236 has a shoulder side wall 238 extending substantially parallel to side wall 224 of body 222 the of pen needle hub 200. Tower 236 has a distal end wall 240 with a distal, axial face 242 forming a skin contact surface. End wall 240 can have a substantially convex shape. Distal face 242 can have a diameter of about 5 to 7 mm. The shoulder has a width to receive the inner shield and a width of about 1-4 mm from the peripheral edge of body 222 and side wall 238 of tower 236. A cavity 286 of the body 222 is defined by the body side wall 238, the shoulder 234, shoulder side wall 238, distal face 260 and proximal open end 226. The cavity 286 is wider at the proximal open end 226 relative to the shoulder 234

A post 244 for supporting a cannula extends inwardly from an inner face 246 of end wall 240 of tower 236 as shown. Post 244 projects inwardly and proximally in the direction of the center axis for supporting cannula 220. Post 244 has an axial passage extending through pen needle hub 200 for receiving the cannula and a conical shaped end 249. A well 248 is formed in end wall 240 at the distal face 242 for receiving an adhesive to couple cannula 220 to pen needle hub 200. In the embodiment shown, cannula 220 extends inwardly a distance for connecting the delivery device and extends outwardly from distal face 242 a distance for piecing the skin of the patient.

The pen needle hub 200 deforms the skin by the insertion force during the insertion and penetration of the cannula by an insertion force normally applied by the patient. In the embodiment shown, the pen needle hub 200 has an inner ring 250 extending from the distal face 242 of tower 236. Inner ring 250 has an axially facing distal face 252 surrounding well 248 and cannula 220 with an inner side surface 254. An outer ring 256 is formed at the outer peripheral edge of tower 236 forming a recess 258 between inner ring 250 and outer ring 256. Outer ring 256 has an axially facing distal face 260 with an inner surface 262 facing inner side surface 254. In the embodiment shown, the surface of recess 258 and the axial faces of inner ring 250 and outer ring 256 have a substantially continuous, concentric radius of curvature and define the skin contact surface of pen needle hub 200. Recess 258 has a depth so that the skin of the patient deflects into the recess and contacts the bottom surface of the recess during needle insertion to deform the skin in a controlled manner. In one embodiment, the radial width of recess is substantially equal to the combined radial width of inner ring 250 and outer ring 256. The axial surface of the distal face of tower 236 has a convex dome shape where inner ring 250 is spaced axially outward relative to outer ring 256 and the axially facing surface of recess 258.

The initial penetration of the cannula 220 by the contact of the inner ring projecting from the tower 236 with the skin of the patient forms a depression in the skin and an initial cannula penetration depth. The surface of the skin then relaxes so that the surface of the skin conforms substantially to the shape of the contact surface formed by outer ring 256 and recess 258 and limits the depth of penetration of the cannula 220. The shape, surface area and height of the contact surface to provide control of the depth of penetration of the cannula during the insertion and penetration force being applied to the injection device.

The side wall 224 of body 222 has an inner surface 266 at the proximal open end 226 of pen needle hub 200. In the embodiment shown, a recess 268 is formed in the inner surface 266. In one embodiment, recess 268 surrounds the circumference of needle hub to form a continuous recess. Recess 268 extends a distance of side wall and terminates at an inclined beveled edge 270. Recess 268 can provide a larger open end to assist in assembling pen needle hub 200 to a delivery device.

The outer surface 272 of side wall 224 includes a plurality of recesses 274 at the upper end forming a scalloped shape. Each recess 274 has a substantially V-shape formed by inclined sides 276 and an open top end 278. The recess 274 can provide the upper portion of the side wall with increased flexibility relative to a surface without recesses. Recess 268 in the bottom end of the side can provide a reduced thickness and flexibility similar to the flexibility provided by the recesses 274.

The face 246 of end wall 240 has a substantially conical shape corresponding to the shape of outer distal face 242. In the embodiment shown, end wall 240 has a substantially uniform thickness. Post 244 has a conical shaped base portion 284 at the inner face 246 that converges to the cylindrical surface of post 244. The conical base 284 also provides stiffness to the end wall 240 to resist deflecting inward during use.

As shown in FIGS. 8 and 9, the pen needle 200 is configured to interdigitate with the male needless connector 120. As shown, the cavity 286 is configured to receive the elongate tip 130 and the collar 140, with the cavity 286 completely surrounding the collar 140. The outer plurality of threads 147 of the outer sidewall surface 146 of the collar 140 are configured to interdigitate with the internal threads 230 of the pen needle 200 until an inner surface of the shoulder 234 abuts the distal end 142 of the collar 140. The conical shaped base portion 284 of the post 244 is at least partially inserted within the lumen 136 of the elongate tip, with the conical shaped base portion 284 forming a first fluid-tight seal 202 the taper 139 of the elongate tip 130. The distal end 132 of the elongate tip 130 forms a second fluid-tight seal 204 with the inner surface of the distal face 260 of the pen needle 200.

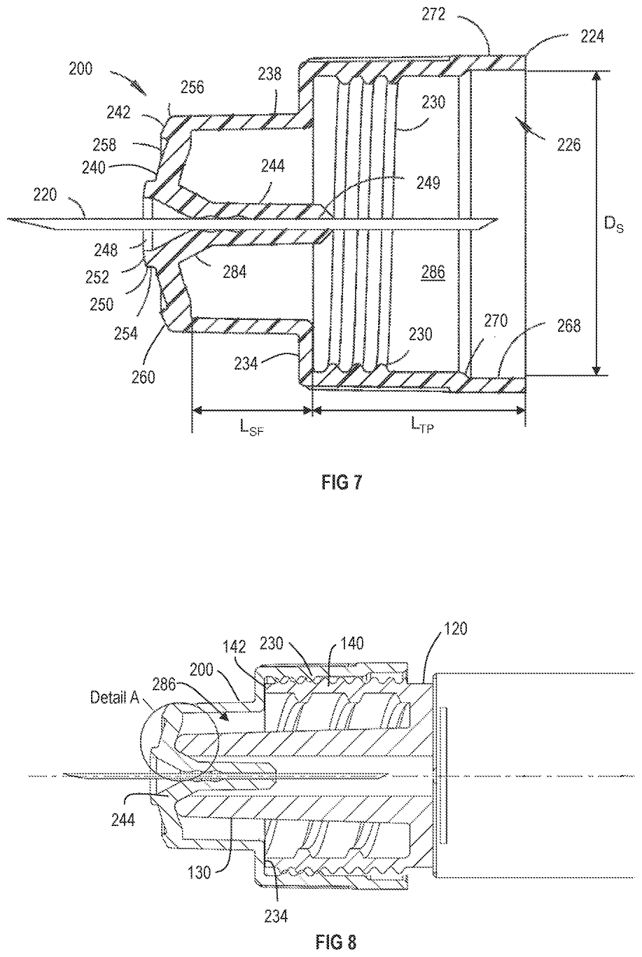

Referring back to FIGS. 1 and 7, a diameter D_{S} of the side wall 224 of the pen needle 200 is configured to accommodate the diameter Dos of the outer sidewall surface 146 of the collar 140, a length L_{SF} from the distal face 260 of the pen needle 200 to the shoulder 234 of the pen needle 200 is configured to accommodate the projection distance M of the distal end 18 of the elongate tip 12 from the distal end 26 of the collar, and a length L_{TP} from the shoulder 234 of the pen needle 200 to the open end 226 of the pen needle 200 is configured to accommodate the projection distance G of the distal end 18 of the elongate tip 12 from the proximal wall 14 in conjunction with the length L_{SF}. As shown, the outer threads 147 of the collar 140 and the internal threads 230 of the pen needle 200 create adequate turns to meet ISO 116-2-2012 torque values of 0.060 Nm to 0.080 Nm. In some embodiments, the outer threads 147 of the collar 140 and the internal threads 230 of the pen needle 200 create more than 2 turns. In some embodiments, the outer threads 147 of the collar 140 and the internal threads 230 of the pen needle 200 create more than 3 turns.

FIG. 10 illustrates an example of improper fitment of the male needless connector 120with the standard pen needle 40 due to the limitations of ISO dimensions of the male needless connector 120 and the standard pen needle 40. FIG. 10 is drawn in proportion and to scale, with dimension projection distance G′ of the distal end 132 of the elongate tip 130 from the proximal base 134 being 7.5 mm, projection distance M′ of the distal end 132 of the elongate tip 130 from the distal end 142 of the collar 140 being 2.1 mm, and length L_{T} of a proximal end 62 of the threads 56 from the distal wall 46 being at 3.7 mm. As shown, only two turns of thread engagement of the threads 56 of the standard needle pen 40 can engage with the plurality of threads 147 of the outer sidewall surface 146 of the male needless connector 120.

In particular a thread engagement length W of the standard needle pen 40 and male needless connector 120 is only 1.2 mm, which is not sufficient to meet ISO torque requirements. As shown, increasing projection distance M′ (distance distal end 132 of the elongate tip 130 from the distal end 142 of the collar 140) would cause even less thread engagement. Decreasing projection distance M′ would bring projection distance M′ out of compatibility with ISO standards. Furthermore, the length L_{T} (proximal end 62 of the threads 56 from the distal wall 46) cannot be increased any further as length L_{T} must be less than 3.7 mm to maintain ISO compatibility. Even if the length T (from an inner surface of the distal wall 46), was increased, the length L_{T} must be less than 3.7 mm to maintain ISO compatibility.

FIG. 11 illustrates a kit 300 comprising the syringe 100 having the male needleless connector 120 integrally molded to the distal end 106 of the barrel 102 of the syringe, the pen needle 200 and the standard female luer needle 70. In some embodiments, each of the syringe 100, pen needle 200 and standard female luer needle 70 are placed in separate pouches 302, 304 and 306 respectively. In some embodiments, the separate pouches 302, 304 and 306 form a single pouch with multiple compartments for each of the syringe 100, pen needle 200 and standard female luer needle 70.

The standard female luer needle 70 comprises a needle hub 72 and a cannula 78. The needle hub 72 has a proximal end 74 and a distal end 76, the cannula 78 extending through the distal end 76 of the needle hub. In some embodiments, the proximal end 74 further comprises a flange 80 configured to thread with the inner threads 145 of the collar 140 of the male needless connector 120. In some embodiments, the proximal end 74 of the needle hub 72 has a conically-shaped cavity (not shown) configured to make an interference fit with the elongate tip 130 of the male needless connector 120.

In some embodiments, the double-ended needle cannula 48 of the pen needle 40 is a small-gauge needle common to pen needles. In some embodiments, the double-ended needle cannula 48 of the pen needle 40 is a small-gauge needle such as a 32 g needle. In some embodiments, the cannula 78 of the standard female luer needle 70 is a standard-gauge needle.

In some embodiments, the cannula 78 of the standard female luer needle 70 is a 25 g needle. Due to the standard female luer needle 70 having a gauge larger in diameter than the pen needle 40, the standard female luer needle 70 is more suitable to withdrawal medicine from vials having a stopper, as the standard female luer needle allows for faster withdrawal.

A method of use of the kit 300 comprises the following steps: attaching the needle hub 72 to the male needless connector 120; inserting the cannula 78 into a stopper of a vial, aspirating medicine from a vial by withdrawing the plunger rod 110 of the barrel 102, removing the cannula 78 from the vial, removing the needle hub 72 from the male needless connector 120, attaching the pen needle 40 to the male needless connector 120, and ejecting medicine by depressing the plunger rod 110.

While the present disclosure has been shown and described with reference to certain exemplary embodiments thereof, it will be understood by those skilled in the art that various changes in form and details may be made therein without departing from the spirit and scope of the embodiments of the present disclosure. Also, the inner and/or the outer housing of the disinfection cap can be single shot molded, or made by other suitable process. Furthermore, any of the features or elements of any exemplary implementations of the embodiments of the present disclosure as described above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of invention.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A male needless connector comprising:
an elongate tip having a distal end and a proximal base, a tapered outer surface and a lumen extending through the elongate tip, the proximal base having a diameter greater than a diameter of the distal end the lumen defining an inner sidewall; and,
a collar having a substantially cylindrical shape and a distal end, the collar partially surrounding the elongate tip, the collar having a cavity forming an inner sidewall surface and an outer sidewall surface, the inner sidewall surface having a diameter greater than the proximal base of the elongate tip, the outer sidewall surface including a plurality of threads for connection to corresponding inner threads of a pen needle,
wherein the inner sidewall surface of the collar includes inner threads for connection to corresponding threads of a female luer connector.

2. The male needless connector of claim 1, wherein a female luer connector is inserted in between the elongate tip and inner sidewall surface.

3. The male needless connector of claim 1, further comprising a taper between the inner sidewall of the elongate tip and the distal end of the elongate tip.

4. The male needless connector of claim 3, wherein the taper has a flat surface.

5. The male needless connector of claim 3, wherein the taper has a flat surface with rounded edges.

6. The male needleless connector of claim 3, wherein the taper has an angle θ relative to a central axis Z of the male needleless connector.

7. The male needless connector of claim 6, wherein the angle θ is in the range of 26 degrees to 28.5 degrees.

8. The male needless connector of claim 3, wherein the taper is configured to create a seal with a post of a pen needle.

9. The male needless connector of claim 1, wherein the male needless connector is compatible with ISO-80369 standards for Male Luer Locks with Fixed Collars.

10. The male needless connector of claim 1, wherein a projection distance G′ of the distal end of the elongate tip from the proximal base is 7.5 mm and a projection distance M′ of the distal end of the elongate tip from the distal end of the collar is 2.1 mm.

11. The male needless connector of claim 1, wherein a projection distance G′ of the distal end of the elongate tip from the proximal base is 7.5 mm and a projection distance M′ of the distal end of the elongate tip from the distal end of the collar is in the range of 4.00 mm to 4.80 mm.

12. The male needless connector of claim 10, wherein the plurality of threads are configured to create more than 2 turns with inner threads of a standard pen needle.

13. A kit comprising:
a syringe having a barrel with an open proximal end, a distal end and a cavity, the male needless connector of claim 1, the male needless connector integrally molded to the distal end of the barrel;
a pen needle hub having a double-ended cannula for coupling to a standard pen needle assembly, the pen needle having a body and side wall forming an open end, the side wall having inner threads for coupling to a standard pen needle assembly; and,
a standard female luer needle having a needle hub and a cannula, the needle hub having a proximal end and a distal end, the cannula extending through the distal end.

14. The kit of claim 13, wherein the body has a distal face with a peripheral edge forming a shoulder, from the shoulder extends a tower away from the open end, the shoulder having a shoulder sidewall and a distal end wall.

15. The kit of claim 13, wherein a post projects proximally from an inner face of the end wall of the pen needle for supporting the double-ended cannula, the post having a conical shaped base portion.

16. The kit of claim 15, wherein a taper between the inner sidewall of the elongate tip and the distal end of the elongate tip is configured to create a seal with the conical shaped base portion of the post.

17. The kit of claim 15, wherein the conical shaped base portion of the post is at least partially inserted within the lumen of the elongate tip creating a seal.

18. The kit of claim 14, wherein the plurality of threads of the outer sidewall surface of the collar are configured to interdigitate with the inner threads of the pen needle until an inner surface of the shoulder abuts the distal end of the collar.

19. The kit of claim 13, wherein the cannula of the standard female luer needle is a standard-gauge needle and the double-ended needle cannula of the pen needle is a small-gauge needle.

20. A method of use of the kit of claim 13 comprising the steps of:
attaching the needle hub to the male needless connector;
inserting the cannula into a stopper of a vial;
aspirating medicine from a vial by withdrawing a plunger rod of the barrel;
removing the needle hub from the male needless connector; and,
attaching the pen needle to the male needless connector;
wherein the pen needle is attached to the male needless connector by threading the inner threads of the pen needle to the plurality of threads of the outer sidewall surface of the collar of the male needless connector.
